(19) United States
(12) Patent Application Publication
(10) Pub. No.: US 2025/0318752 A1
(43) Pub. Date: Oct. 16, 2025

(54) AUDIO DETECTION SYSTEMS AND RELATED METHODS OF DETECTING A CONDITION OF THE EAR

(71) Applicant: Sonorous NV, Lake Forest, CA (US)

(72) Inventors: Charles E. Stout, Loma Linda, CA (US); Dave Ferrera, Coto De Caza, CA (US); Jake Le, Lake Forest, CA (US)

(21) Appl. No.: 19/174,643

(22) Filed: Apr. 9, 2025

Related U.S. Application Data
(60) Provisional application No. 63/632,338, filed on Apr. 10, 2024.

(51) Int. Cl. A61B 5/12; A61B 5/00

(52) U.S. Cl. CPC A61B 5/128; A61B 5/682; A61B 5/7405; A61B 5/742; A61B 5/7475; A61B 2560/0462; A61B 2562/0204; A61B 2562/227

(57) ABSTRACT

An audio detection system includes a mouthpiece configured to detect vibrations occurring at a subject's head, generate a signal based on the vibrations, and transmit the signal. The audio detection system also includes a signal processing module configured to receive the signal from the mouthpiece as an audio input signal, generate an output signal based on the audio input signal, and transmit the output signal. A method of detecting an abnormal condition experienced by a subject includes detecting vibrations within the subject's head at a mouthpiece positioned within the subject's mouth, generating a signal based on the vibrations at the mouthpiece, transmitting the signal from the mouthpiece to a signal processing module, receiving the signal as an audio input signal at the signal processing module, generating an output signal based on the audio input signal at the signal processing module, and transmitting the output signal.

### CROSS REFERENCE TO RELATED APPLICATION

This application claims priority to U.S. Provisional Patent Application No. 63/632,338, filed on Apr. 10, 2024. The content of this provisional application is incorporated herein by reference in its entirety.

### TECHNICAL FIELD

This disclosure relates to audio detection systems and associated sensors capable of sensing vibrations associated with a condition of the ear and an underlying pathology. This disclosure further relates to methods of detecting and diagnosing the condition and the underlying pathology.

### BACKGROUND

Pulsatile tinnitus (e.g., sometimes referred to as vascular tinnitus) is a condition in which a person hears rhythmic thumping, whooshing, throbbing, or other sounds that are synchronous with the person's heartbeat. A person may hear such sounds in one or both ears. Pulsatile tinnitus can significantly reduce a person's quality of life. In some cases, pulsatile tinnitus results from cerebral venous sinus stenosis (CVSS), which is a vascular pathology (e.g., a vascular disease) that results in obstruction of the flow of blood out of the cerebral dural venous sinuses. In some cases, pulsatile tinnitus alternatively or additionally results from one or more other vascular pathologies.

### SUMMARY

This disclosure relates to audio detection systems designed to detect an abnormal ear condition experienced by a patient and an underlying vascular pathology. Such an audio detection system may include a mouthpiece by which vibrations can be detected within the patient's head via bone conduction and a signal processing module that generates an output signal based on the detected vibrations. The signal processing module can transmit the output signal to a display screen and/or a storage. The signal processing module can also convert the output signal to an analog audio output signal and transmit the analog audio output signal to an output device that receives and provides the analog audio output signal to a listener. This disclosure further relates to methods of detecting and diagnosing the abnormal condition and underlying vascular pathology using the audio detection system.

Such an audio detection system provides a robust tool for accurately detecting pulsatile tinnitus that results from cerebral venous sinus stenosis (CVSS). The mouthpiece of the audio detection system may be embodied as an ergonomic bite block that facilitates comfortable holding of the mouthpiece between the patient's teeth for a required time period during which the vibrations are sensed at the mouthpiece. Moreover, due to the mouthpiece's ability to take advantage of bone conduction within the patient's head (e.g., via the patient's teeth), the audio detection system allows a diagnostic investigation without subjecting the patient to an invasive procedure. The audio detection system is also capable, advantageously, of outputting data (e.g., an audio signal and corresponding visual data) in a manner that allows perception (e.g., listening and seeing, respectively) by both the patient and a clinician.

In one aspect, an audio detection system includes a mouthpiece configured to detect vibrations occurring at a subject's head, generate a signal based on the vibrations, and transmit the signal. The audio detection system also includes a signal processing module configured to receive the signal from the mouthpiece as an audio input signal, generate an output signal based on the audio input signal, and transmit the output signal.

Embodiments may include one or more of the following features.

In some embodiments, the mouthpiece includes a bite block that is configured to be held between two or more of the subject's teeth and configured to conduct the vibrations from the two or more of the subject's teeth.

In some embodiments, the mouthpiece includes a bone conduction device.

In some embodiments, the mouthpiece includes a vibration sensor.

In some embodiments, the vibration sensor is configured to detect the vibrations as an analog signal.

In some embodiments, the vibration sensor is formed on an integrated circuit.

In some embodiments, the mouthpiece includes an electrical cable that electrically connects the vibration sensor to the signal processing module.

In some embodiments, the output signal has a frequency within a range of about 100 Hz to about 10,000 Hz.

In some embodiments, the signal processing module includes a benchtop console.

In some embodiments, the signal processing module includes a handheld console.

In some embodiments, the output signal includes a digital output signal.

In some embodiments, the signal processing module is configured to store the digital output signal as one or more recorded sounds.

In some embodiments, the signal processing module is configured to transmit the digital output signal to a display device for display in graphical form.

In some embodiments, the signal processing module is configured to convert the digital output signal to an analog audio output signal and transmit the analog audio output signal to an output device.

In some embodiments, the audio detection system further includes the output device.

In some embodiments, the output device includes a headphone.

In some embodiments, the output device includes a speaker.

In some embodiments, the signal processing module is configured to generate an indicator associated with the audio output signal and corresponding to an abnormal condition of the ear that is secondary to an underlying vascular pathology.

In some embodiments, the abnormal condition is pulsatile tinnitus.

In some embodiments, the underlying vascular pathology is cerebral venous sinus stenosis (CVSS).

In another aspect, a method of detecting an abnormal condition experienced by a subject includes detecting vibrations within the subject's head at a mouthpiece positioned within the subject's mouth, generating a signal based on the vibrations at the mouthpiece, transmitting the signal from the mouthpiece to a signal processing module, receiving the signal as an audio input signal at the signal processing module, generating an output signal based on the audio input signal at the signal processing module, and transmitting the output signal.

Embodiments may include one or more of the following features.

In some embodiments, the mouthpiece includes a bite block that is held between two or more of the subject's teeth and configured to conduct the vibrations from the two or more of the subject's teeth.

In some embodiments, the mouthpiece includes a vibration sensor, and the method further includes detecting the vibrations as an analog signal at the vibration sensor.

In some embodiments, the output signal includes a digital output signal.

In some embodiments, the method further includes storing the digital output signal as one or more recorded sounds and/or transmitting the digital output signal to a display screen and displaying a representation of the digital output signal on the display screen for observation by a viewer.

In some embodiments, the method further includes converting the digital output signal to an analog audio output signal and transmitting the analog audio output signal to an output device for perception by a listener.

In some embodiments, the output device includes a headphone.

In some embodiments, the output device includes a speaker.

In some embodiments, the method further includes outputting an indicator associated with the output signal to a display screen.

In some embodiments, the indicator corresponds to the abnormal condition and a vascular pathology that underlies the abnormal condition.

In some embodiments, the abnormal condition is pulsatile tinnitus.

In some embodiments, the vascular pathology is cerebral venous sinus stenosis (CVSS).

In some embodiments, the indicator includes one or more frequencies of the output signal.

In another aspect, a method of diagnosing a subject with an abnormal condition that is secondary to a vascular pathology includes instructing the subject to bite down a mouthpiece disposed within the subject's mouth, perceiving an output signal generated by a signal processing module based on vibrations detected by a vibration sensor within the mouthpiece, and identifying the abnormal condition and the vascular pathology based on an analysis of the output signal.

Embodiments may include one or more of the following features.

In some embodiments, the abnormal condition is pulsatile tinnitus, and the vascular pathology is cerebral venous sinus stenosis (CVSS).

In some embodiments, the vibrations are conducted through one of more of the subject's skull bones, through the subject's teeth, and through at least a portion of the mouthpiece.

In some embodiments, the output signal includes an analog audio output signal, and the method further includes listening to the analog audio output signal.

In some embodiments, the audio output signal includes a pulsating sound that corresponds to an objective sound associated with the vibrations.

In some embodiments, the method further includes viewing an indicator associated with the output signal.

In some embodiments, the indicator includes one or more frequencies of the audio output signal.

In another aspect, a pulsatile tinnitus diagnosis system includes a bite block including a vibration sensor that is configured to detect objective sounds as vibrations occurring at a patient's head, generate a signal based on the vibrations, and transmit the signal. The pulsatile tinnitus diagnosis system further includes a signal processing console configured to receive the signal from the bite block, generate an output signal based on the signal, store the output signal as one or more recorded sounds, and transmit the output signal. The pulsatile tinnitus diagnosis system further includes a headphone configured to receive an analog audio output signal from the signal processing console that corresponds to the output signal.

In another aspect, an audio detection system includes a mouthpiece containing a vibration sensor that generates a signal based on the vibrations generated by turbulent blood flow within a cerebral venous sinus and a signal processing module configured to receive the signal from the mouthpiece as an audio input signal and to generate an output signal based on the audio input signal.

The details of one or more embodiments are set forth in the accompanying drawings and description. Other features, aspects, and advantages of the embodiments will become apparent from the description, drawings, and claims.

### DESCRIPTION OF DRAWINGS

FIG. 1 is a schematic diagram of an example audio detection system.

FIG. 2 is a top perspective view of an example mouthpiece of the audio detection system of FIG. 1.

FIG. 3 is a side perspective view of the mouthpiece of FIG. 2.

FIG. 4 is a front perspective view of the example mouthpiece, an example signal processing console, and an example output device of the audio detection system of FIG. 1.

FIG. 5 is an example block diagram of the signal processing console of FIG. 4 and the example signal processing console of FIG. 6.

FIG. 6 is a front perspective view of the example mouthpiece, an example signal processing console, and the example output device of the audio detection system of FIG. 1.

FIG. 7 is a front view of a user interface of the signal processing console of FIG. 6.

FIG. 8 is a schematic diagram of an example audio detection system.

FIG. 9 is an example block diagram of an example signal processing module of the audio detection system of FIG. 8.

FIG. 10 is a flow chart illustrating an example method of detecting an abnormal condition experienced by a subject.

FIG. 11 is a flow chart illustrating an example method of diagnosing a subject with an abnormal condition that is secondary to a vascular pathology.

### DETAILED DESCRIPTION

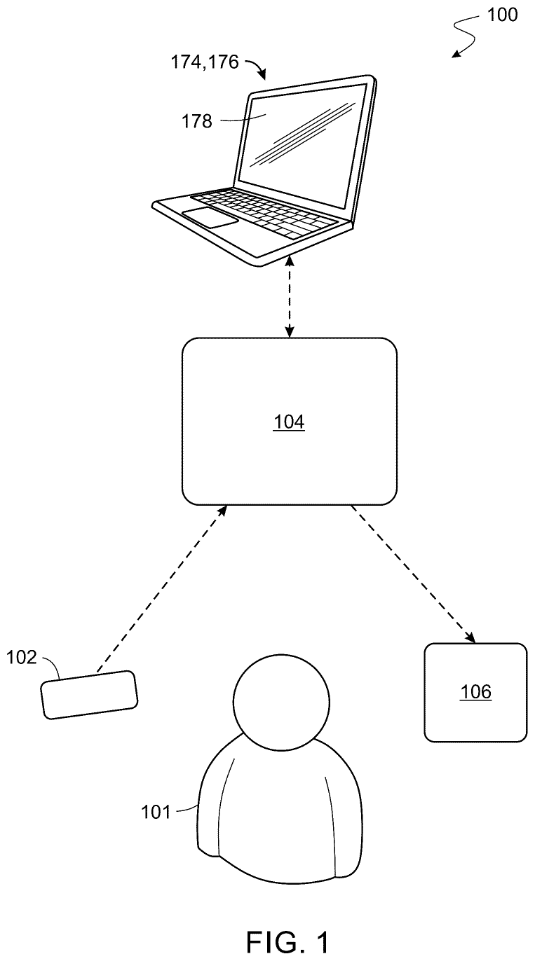

FIG. 1 illustrates an example audio detection system 100 that is used to detect, identify, diagnose, or otherwise investigate a condition of one or both ears of a patient 101 or other subject of the audio detection system 100. In some examples, the condition is an abnormal or otherwise bothersome condition, such as pulsatile tinnitus. The pulsatile tinnitus results from (e.g., is secondary to) an underlying vascular pathology that is experienced by the patient 101. The vascular pathology may be identifiable by the audio detection system 100 or identifiable by a clinician utilizing data that is output from the audio detection system 100. In some cases, the vascular pathology is cerebral venous sinus stenosis (CVSS).

The audio detection system 100 includes a mouthpiece 102, a signal processing module 104, and an audio output device 106. The mouthpiece 102 captures (e.g., detects or senses) objective sounds within the patient 101 as an audio input signal, processes the audio input signal, and sends a processed (e.g., digital) audio signal to the signal processing module 104. The signal processing module 104 receives the processed audio signal from the mouthpiece 102 as an input signal to the signal processing module 104, further processes the input signal, outputs an audio output signal to the output device 106, and displays a visual representation corresponding to the audio output signal. The output device 106 receives the audio output signal from the signal processing module 104 and outputs the audio output signal for perception (e.g., hearing) by an ear of a listener (e.g., the patient 101, a clinician, or another individual). The visual representation is displayed on a screen of the signal processing module 104 for viewing by the listener or another individual.

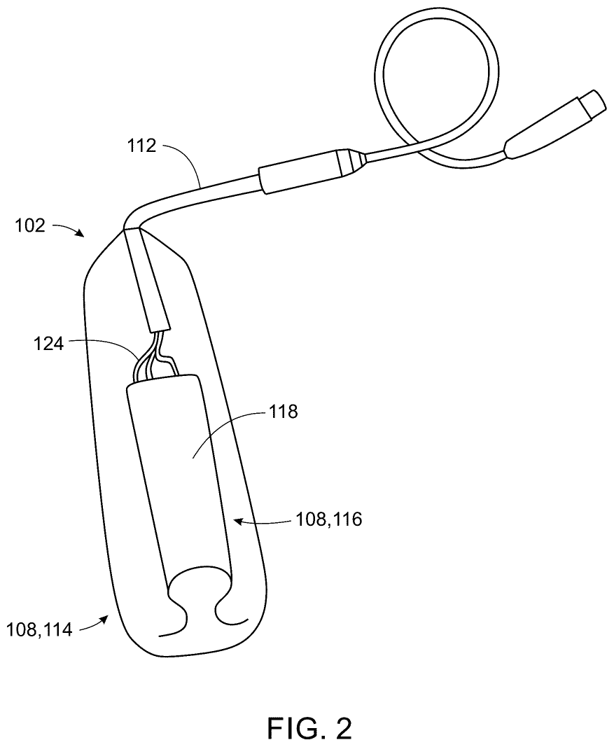

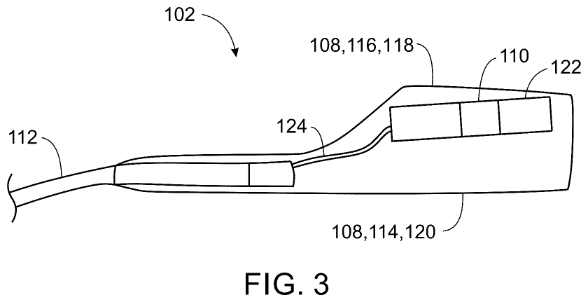

Referring to FIGS. 2 and 3, in some embodiments, the mouthpiece 102 is provided as a bite block that is designed to be inserted within the patient's mouth, to be bitten by the patient's teeth, and to detect vibrations experienced by the patient's teeth. Accordingly, the mouthpiece 102 is capable of withstanding the compressive force applied by the bite of the patient's upper and lower teeth. That is, the mouthpiece 102 can maintain its functional (e.g., mechanical, electrical, and other) integrity upon being bitten by the patient. Additionally, the mouthpiece 102 is a disposable component such that the mouthpiece 102 is typically discarded after being used for a single diagnostic inquiry or after being used for multiple diagnostic inquiries that make up a single diagnostic session.

The mouthpiece 102 includes a housing 108, a vibration sensor 110 that is embedded within the housing 102, and an electrical cable 112 that transmits signals between the vibration sensor 110 and the signal processing module 104 when the cable 112 is connected to the signal processing module 104. The housing 108 is an elongate, rigid component that is ergonomically shaped to provide for a comfortable bite by the patient. The housing 108 includes a substantially flat base 114 and a compartment wall 116 that surrounds the vibration sensor 110. The compartment wall 116 may have a substantially circular cross-sectional shape (as shown), or a different, non-circular cross-sectional shape. The patient's bite is expected to contact the mouthpiece 102 along a top surface 118 of the compartment wall 116 and a bottom surface 120 of the flat base 114. In some examples, the patient's teeth may exert a force of about 50 N to about 200 N on the mouthpiece 102 during a bite.

In some embodiments, the housing 108 is a molded component. In some embodiments, the housing 108 is made of one or more ultra-rigid materials that can conduct vibrations without substantial signal loss (e.g., dampening). In some embodiments, the housing 108 is made of a material that is generally safe, non-toxic, and neither biologically nor otherwise undesirable. The housing 108 is made of one or more materials. Example materials from which the housing 108 may be made include polyetherimide, polyetheretherketone (PEEK), polycarbonate, high-density polyethylene (HDPE), UV-cured resins (e.g., methacrylate, polyurethane acrylates, epoxy-based UV resin, etc.), thermoset plastics that have been chemically cured at room temperature (e.g., bisphenol-A epoxy, polymethyl methacrylate, silicone room temperature vulcanizing, etc.), and thermoplastics, such as dental mold that softens when heated (e.g., polycaprolactone, polyethylene terephthalate glycol, ABS-based hot-melt resin, etc.).

The housing 108 may be transparent, translucent, or opaque. In some embodiments, the housing 108 has a total length of about 3 cm to about 10 cm (e.g., about 7 cm). In some embodiments, the housing 108 has a total width of about 1 cm to about 2 cm (e.g., about 1.5 cm). In some embodiments, the compartment wall 116 of the housing 108 has a total width of about 0.5 cm to about 1.0 cm (e.g., about 0.75 cm) and a length of about 2 cm to about 4 cm (e.g., about 3 cm). In some embodiments, the housing 108 has a total height of about 1 cm to about 2 cm (e.g., about 1.5 cm). In some embodiments, the electrical cable 112 is made of polyolefin heat shrink tubing. In some embodiments, the electrical cable 112 has a length of about 100 cm to about 600 cm (e.g., about 300 cm).

In some embodiment, the vibration sensor 110 is formed on a flexible, integrated circuit 122 within the compartment wall 116 of the housing 108. The vibration sensor 110 is designed to detect objective sounds within the patient's head as vibrations. In some examples, such vibrations are generated by turbulent blood flow within cerebral venous sinuses that are experiencing stenosis. Such vibrations have been conducted through one or more of the patient's skull bones and through the patient's teeth that bite down on the mouthpiece 102. With respect to bone conduction, the patient's teeth may be considered extensions of the patient's bones. Owing to conduction of the vibrations through the patient's skull bones, subsequently through the patient's teeth, and subsequently through the housing 108 and to the vibration sensor 110, the mouthpiece 102 may be considered a bone conduction device.

The vibration sensor 110 detects the vibrations as an analog audio signal. Accordingly, the mouthpiece 102 (e.g., including vibration sensor 110 disposed therein) may be considered a microphone. In some embodiments, the vibration sensor 110 converts the analog audio signal into a digital signal within a frequency range of about 100 Hz to about 10,000 Hz, thereby producing a digital audio signal. The vibration sensor 110 includes one or more wires 124 that transmit the digital audio signal to the electrical cable 122.

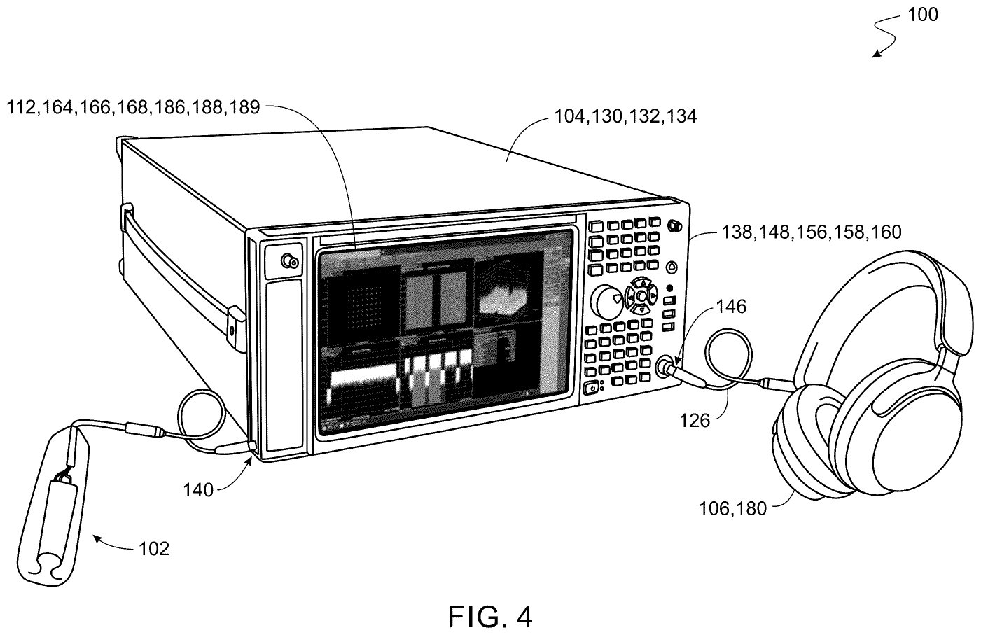

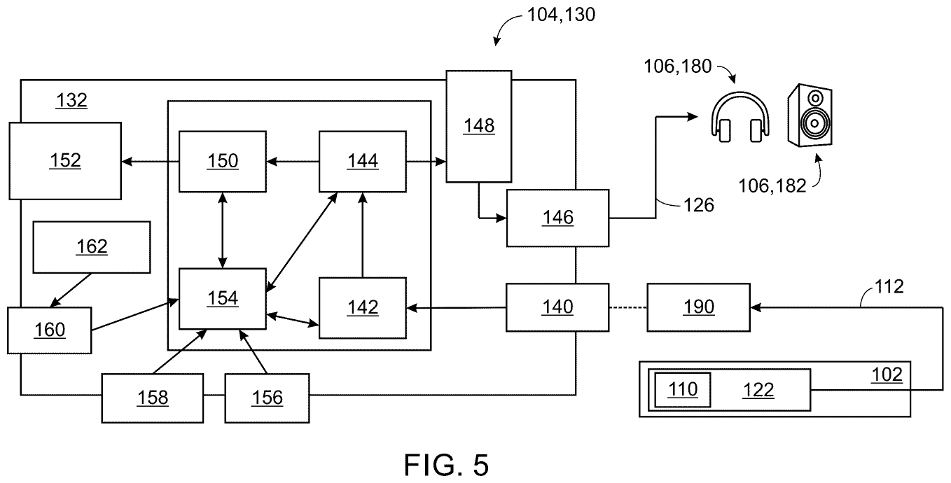

Referring to FIGS. 1 and 4, the signal processing module 104 may be embodied as a signal processing console 130 that receives the digital audio signal from the mouthpiece 102. In some embodiments, the console 130 is a table-top (e.g., a benchtop) console. The console 130 filters out noise within the digital audio signal to isolate the sound frequencies within the digital audio signal and subsequently amplifies the filtered, digital audio signal. The console 130 includes a housing 132 that contains or supports one or more processors and other internal hardware components for executing the various console functionalities, as illustrated in the block diagram in FIG. 5. In some embodiments, the housing 132 has a length of about 20 cm to about 30 cm (e.g., about 25 cm), a width of about 15 cm to about 25 cm (e.g., about 20 cm), and a height of about 10 cm to about 15 cm (e.g., about 12.5 cm). The console 130 also includes a display screen 136 and several user input controls 138 for controlling various functionalities of the console.

Referring to FIGS. 4 and 5, the console 130 includes an audio-input connector 140 that receives the digital audio signal from the vibration sensor 110 as an audio input signal, an amplifier 142 (e.g., with an auto gain control circuit) that receives the digital audio signal from the connector 140, and a digital signal processor (DSP) 144 that receives the digital audio signal (e.g., a gain-adjusted signal) from the amplifier 142. In some embodiments, an intermediate audio-input connector 190 couples the vibration sensor 110 to the connector 140. The DSP 144 analyzes, filters, isolates, and converts the digital audio signal to an output signal (e.g., a processed, digital signal) that can be transmitted for storage and transmitted for display on the display screen 136. The DSP 144 can also convert the output signal to an analog audio output signal and transmit the analog audio output signal to the output device 106 through an audio-output connector 146.

The console 130 includes an externally accessible volume controller 148 (e.g., a knob, dial, or other type of control component) by which a user (e.g., the patient 101, a clinician, or another user) can adjust a level (e.g., a volume or other level) of the output signal. The console 130 further includes a storage buffer 150 that receives the output signal from the DSP 144 and a secure digital (SD) card 152 that receives the output signal from the storage buffer 150 and stores the output signal thereon as one or more recorded sounds. The SD card 152 can be removed from the console 130 and installed in a computer for subsequent access to its stored data (e.g., the output signal).

Additionally, the console 130 includes a microcontroller unit (MCU) 154 that controls various functionalities of the console 130. The MCU 154 is capable of transmitting signals to and receiving signals from the storage buffer 150, the DSP 144, and the amplifier 142. Furthermore, in addition to the volume controller 148, the user input controls 138 also include a capture switch 156 by which the user can cause the console 130 to start or stop capturing audio signals from the mouthpiece 102, an equalization (EQ) mode switch 158 by which the user can select a frequency range (e.g., high, low, or wide) whose volume level should be adjusted (e.g., amplified), and a power switch 160 for powering the console 130 on and off. As illustrated in FIG. 5, the capture switch 156, EQ mode switch 158, and power switch 160 are configured to send signals to the MCU 154. The console 130 also includes one or more batteries 162 for powering the console 130 and which are accordingly electrically coupled to the power switch 160.

The display screen 136 of the console 130 can display one or more indicators associated with signals inputted to and outputted from the console 130. In some embodiments, the indicators include three indicators 166 that respectively correspond to the frequency ranges that are selectable via the EQ mode switch 158, an indicator 168 that indicates the capture of signals from the vibration sensor 110, and one or more indicators 186 (e.g., one or more numbers, one or more symbols, or one or more other graphical displays, and/or one or more other types of indicator) that corresponds to an amplitude or a frequency or frequency range of the audio output signal. For example, in some embodiments, the one or more indicators 186 are displayed as one or more frequency components of the audio output signal on one or more spectra graphs 188 and/or as one or more waveforms 189 corresponding to the audio output signal. In some examples, the display screen 136 and the user controls 138 together form a user interface 164 of the console 130.

In some embodiments, the audio detection system 100 additionally or alternatively includes software 184 installed on a computer 174 that is electronically coupled (e.g., in a wired or wireless manner) to the console 130 and which has a display screen that can display the one or more indicators 166, 168, 186 and the one or more graphs 188, 189. The computer 174 includes one or more processors 176 and a display screen 178. In some embodiments, the computer 174 can alternatively perform one or more of the above-discussed functionalities of the console 130.

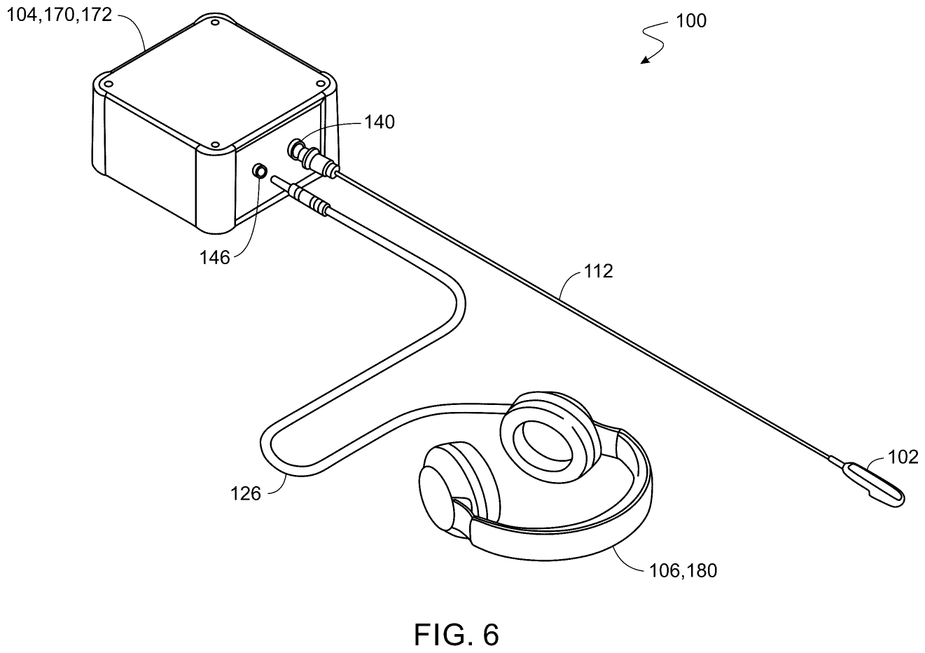

Referring to FIGS. 1 and 6, in some embodiments the signal processing module 104 may alternatively be embodied as a handheld signal processing console 170 or another type of signal processing module that is relatively portable. In some embodiments, the signal processing console 170 is functionally equivalent to the signal processing console 130 and accordingly includes a housing 172 that contains or otherwise supports all of the components and functionalities discussed above with respect to FIGS. 4 and 5, including the audio-input connector 140, amplifier 142, DSP 144, audio-output connector 146, volume controller 148, storage buffer 150, SD card 152, MCU 154, capture switch 156, EQ mode switch 158, power switch 160, and battery pack 162. Furthermore, the console 170 is designed to be electronically coupled (e.g., in a wired or wireless manner) to the computer 174. In some embodiments, the housing 172 of the console 170 has a length of about 15 cm to about 25 cm (e.g., about 20 cm), a width of about 7 cm to about 20 cm (e.g., about 15 cm), and a height of about 3 cm to about 5 cm (e.g., about 4 cm).

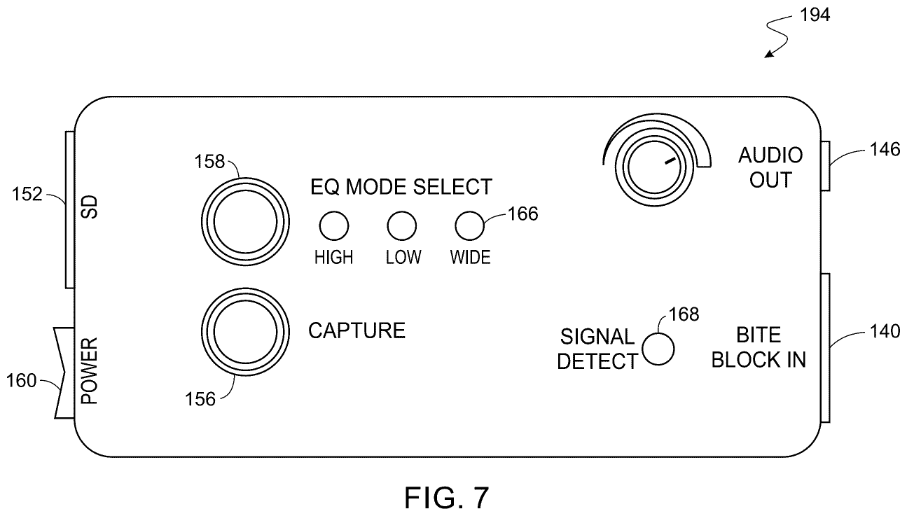

FIG. 7 illustrates a front view of one version of a user interface 194 of the console 130. In some embodiments, the user interface 194 is provided on a side of the console 130. In some embodiments, the user interface 194 provides access to the audio-input connector 140, the audio-output connector 146, the power switch 160, the SD card 152, the capture switch 156, and the EQ mode switch 158. The user interface 194 also provides the three indicators 166 that respectively correspond to the frequency ranges that are selectable via the EQ mode switch 158, as well as the indicator 168 that is activated (e.g., illuminated) while a signal is captured from the vibration sensor 110. In some embodiments, the signal processing console 130 is designed to be electronically coupled (e.g., in a wired or wireless manner) to the computer 174. The display screen 178 of the computer 174 can display one or more graphs or other indicators that correspond to the signals that are inputted to and outputted from the console 130.

Referring again to FIG. 5, in some embodiments, the output device 106 is provided as one or more headphones 180 (e.g., a set of headphones) at which the user can listen to the analog audio signals outputted from the signal processing module 104 (e.g., the console 130 or the console 170). In other embodiments, the output device 106 includes one or more speakers 182 by which the user can listen (e.g., or by which multiple users can simultaneously listen) to the analog audio signals outputted from the signal processing module 104. The output device 106 may be coupled to the signal processing module 104 via an electrical cable 126 that is connected to the audio-output connector 146.

During operation of the audio detection system 100, the mouthpiece 102 is inserted within a patient's mouth (e.g., by the patient, a clinician, or another individual), and the patient bites down on the mouthpiece 102. The patient typically bites down on the mouthpiece 102 for a duration of about 30 seconds to about 90 seconds. While the mouthpiece 102 is disposed between the patient's upper and lower teeth and experiences compression forces by the patient's bite, vibrations caused by turbulent blood flow (e.g., resulting from venous sinus stenosis or other vascular abnormalities) can be heard as an objective sound at the patient's ears. Those same vibrations are detected via bone conduction by the vibration sensor 110 within the mouthpiece 102.

The vibration sensor 110 converts the vibrations (e.g., initially in analog form) into a digital audio signal with frequencies in a range of about 100 Hz to about 10,000 Hz. The vibration sensor 110 transmits the digital audio signal, via the wire 124 and electrical cable 112, to the signal processing module 104 (e.g., the console 130 or the console 170). The signal processing module 104 isolates and amplifies the sound frequencies within the signal and converts the isolated, amplified sound frequencies into an output signal. The signal processing module 104 then converts the output signal to an analog audio output signal and outputs the analog audio output signal to the output device 106 (e.g., the headphones 180 or the speaker 182).

Based on a frequency (e.g., a pitch) associated with the output signal, one or more listeners (e.g., the patient, a physician, or another clinician) can diagnose or otherwise identify pulsatile tinnitus and an underlying vascular pathology that causes the pulsatile tinnitus. For example, the frequency may be indicated to the user as one or more indicators 186 displayed as one or more frequency components of the output signal on one or more spectra graphs 188 that are displayed on the console display screen 136 or on the computer display screen 178. In some embodiments, the output signal may additionally be presented in association with one or more waveforms 189 corresponding to the output signal. Example conditions or pathologies that may be diagnosed using the audio detection system 100 include pulsatile tinnitus secondary to CVSS, pulsatile tinnitus secondary to arteriovenous malformation, pulsatile tinnitus secondary to arteriovenous fistula. In some examples, pulsatile tinnitus secondary to CVSS may be associated with a frequency range of about 100 Hz to about 1,000 Hz. In some examples, pulsatile tinnitus secondary to arteriovenous malformation may be associated with a frequency range of about 1,000 Hz to about 2,000 Hz. In some examples, pulsatile tinnitus secondary to arteriovenous fistula may be associated with a frequency range of about 1,000 Hz to about 2,000 Hz. In some examples, any of pulsatile tinnitus secondary to CVSS, pulsatile tinnitus secondary to arteriovenous malformation, pulsatile tinnitus secondary to arteriovenous fistula, or other conditions and pathologies may be associated with a frequency range of about 100 Hz to about 10,000 Hz.

Provision of an output signal that is related to the sensed vibrations and provision of an associated frequency indicator each enable objective diagnosis and characterization of pulsatile tinnitus and an associated, underlying pathology experienced by the patient. This objectivity provides an improved diagnostic approach as compared to subjective techniques conventionally used to diagnose such conditions, such as where a patient describes the sound that the patient hears or where a physician listens to the sound using a stethoscope.

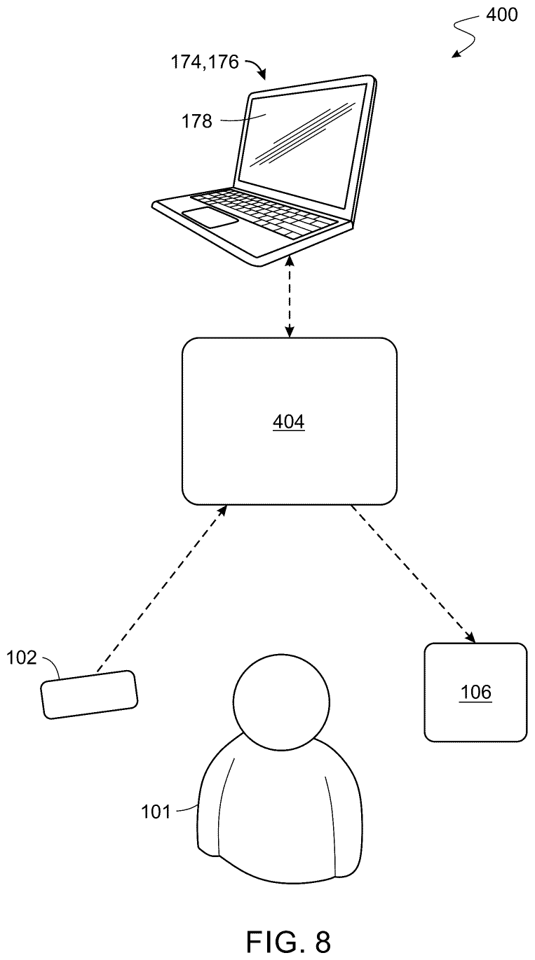

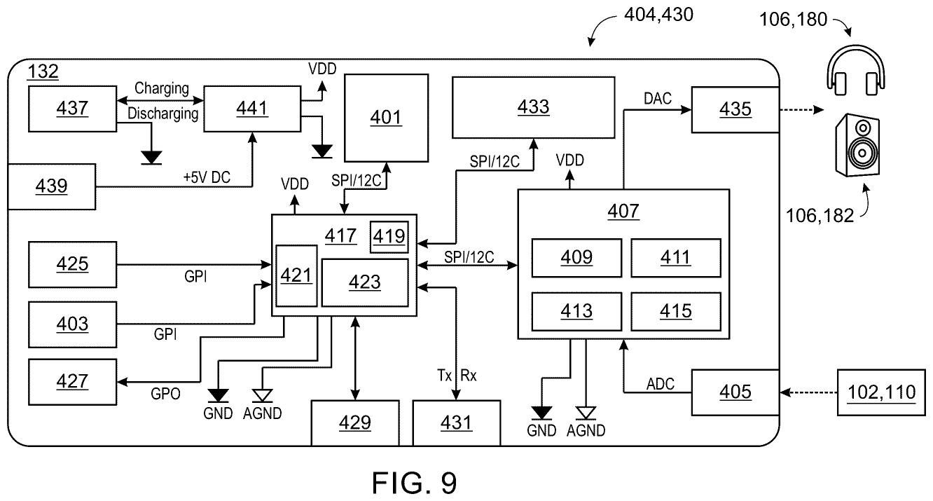

Referring to FIGS. 8 and 9, in some embodiments, an audio detection system 400 that is otherwise similar in construction and function to the audio detection system 100 includes a signal processing module 404 instead of the signal processing module 104. Accordingly, the audio detection system 400 includes also the mouthpiece 102 and the audio output device 106 that are described above with respect to the audio detection system 100.

Referring to FIG. 9, the signal processing module 404 may be embodied as a signal processing console 430 that receives the digital audio signal from the mouthpiece 102. In some embodiments, the console 430 is a table-top (e.g., a benchtop) console. The console 430 filters out noise within the digital audio signal to isolate the sound frequencies within the digital audio signal and subsequently amplifies the filtered, digital audio signal. The console 430 includes the housing 132, which contains or supports one or more processors and other internal hardware components for executing the various console functionalities, as illustrated in the block diagram of FIG. 9. The console 430 also includes a display screen 401 and several externally accessible user input controls 403 for controlling various functionalities of the console 430. Example user input controls 403 include gain selection, frequency selection, volume control, capture control, equalization, and others.

The signal processing console 430 includes an audio-input connector 405 that receives the digital audio signal from the vibration sensor 110 as an audio input signal to the signal processing console 430. The console 430 also includes an audio codec integrated circuit (IC) 407 (e.g., an ADAU1772) that receives the audio input signal (e.g., a digital signal) from the connector 405. In some embodiments, the IC 407 converts the audio input signal into a signal within a frequency range of about 100 Hz to about 10,000 Hz. The IC 407 includes functionalities for filtering 409, level control and monitoring 411, signal pre-processing 413, and gain control 415. For example, the IC 407 pre-processes the audio input signal by isolating and amplifying certain sound frequencies within the signal. The console 430 also includes an MCU 417 that receives the pre-processed signal from the IC 407 via a serial peripheral interface (SPI) embodied as a four-wire serial communication protocol) and an integrated circuit embodied as a two-wire serial communication protocol (I2C). The MCU 417 post-processes the digital signal based on various user inputs received at the console 430 to generate an output signal (e.g., a digital output signal). The MCU 417 includes functionalities for equalization 419, buffering 421, and signal post-processing 423.

The MCU 417 is also capable of transmitting signals to and/or receiving signals from several other components of the signal processing console 430. Such components include a power switch 425 by which the console 430 can be powered on and off via a general purpose input (GPI), the various user input controls 403 via a GPI, status indicators 427 (e.g., status light emitting diodes, LEDs) that serve as indicators via a general purpose output (GPO), a serial wire debug (SWD) connector 429, a universal asynchronous receiver (Rx)/transmitter (Tx) (UART) 431 for debugging, and a data storage/memory 433 (e.g., a micro SD card or a flash memory) via an SPI/I2C. The MCU 417 is also capable of transmitting the digital output signal to the display screen 401 via an SPI/I2C.

The MCU 417 transmits the output signal (e.g., a digital output signal) to the audio codec IC 407, which then converts the output signal to an analog audio output signal. The IC 407 transmits the analog audio output signal to the output device 106 through an audio-output connector 435 (e.g., a 3.5 mm audio-output connector). The MCU 417 is also capable of transmitting the output signal (e.g., a digital output signal) to the display screen 401, which displays one or more visual representations of the output signal (e.g., in graphical or other visual form). In some implementations, the visual representations are provided as one or more indicators 186 that are displayed as one or more frequency components of the output signal on one or more spectra graphs 188. In some implementations, the visual representations are alternatively or additionally provided as one or more waveforms 189 corresponding to the output signal. The MCU 417 is also capable of transmitting the output signal (e.g., a digital output signal) to the data storage/memory 433, where the output signal is stored thereon as one or more recorded sounds.

The signal processing console 430 also includes one or more batteries 437 for powering the console 430 and which are accordingly electrically coupled to the power switch 425. The console 430 also includes a USB connector 439 for receiving a USB drive. Both the one or more batteries 437 and the USB connector 439 are electronically coupled to a power management integrated circuit (PMIC) 441. Each of the PMIC 441, MCU 417, and audio codec IC 407 have connections or other hardware for drain voltage (e.g., VDD) and grounding (e.g., GND and/or AGND).

As discussed above, the display screen 401 of the signal processing console 430 can display one or more indicators associated with signals inputted to and outputted from the console 430. In some embodiments, the indicators include three indicators that respectively correspond to the frequency ranges that are selectable via an EQ mode switch, an indicator that indicates the capture of signals from the vibration sensor 110, and one or more indicators (e.g., one or more numbers, one or more symbols, or one or more other graphical displays, and/or one or more other types of indicator) that corresponds to an amplitude or a frequency or frequency range of the digital output signal (e.g., and of the converted analog audio output signal). For example, in some embodiments, the one or more indicators are displayed as one or more frequency components of the output signal on one or more spectra graphs and/or as one or more waveforms corresponding to the output signal. Based on a frequency (e.g., a pitch) associated with the output signal, one or more listeners and/or viewers (e.g., the patient, a physician, or another clinician) can diagnose or otherwise identify pulsatile tinnitus and an underlying vascular pathology that causes the pulsatile tinnitus, as discussed above with respect to the audio detection system 100 and the above-discussed underlying vascular pathologies.

In some examples, the display screen 401 and the user controls 403 together form a user interface of the console 430. In some embodiments, the audio detection system 400 additionally or alternatively includes software (e.g., such as the software 184) installed on the computer 174, which may be electronically coupled (e.g., in a wired or wireless manner) to the console 430. In some embodiments, the computer 174 can alternatively perform one or more of the above-discussed functionalities of the console 430.

As discussed above with respect to the signal processing console 130, the signal processing console 430 includes a user interface (e.g., similar to the user interface 194) that may be provided on a side of the console 430. The user interface provides access to the audio-input connector 405, audio-output connector 435, SWD connector 429, UART 431, status indicators 427, user input controls 403, power switch 425, and USB connector 439.

In some embodiments, the signal processing module 404 may alternatively be embodied as a handheld signal processing console (e.g., such as a console similar to that of the console 170) or another type of signal processing module that is relatively portable. In some embodiments, the handheld signal processing console is functionally equivalent to the signal processing console 430 and includes the housing 172, which contains or otherwise supports all of the components and functionalities discussed above with respect to FIG. 9. Furthermore, the handheld console is additionally or alternatively designed to be electronically coupled (e.g., in a wired or wireless manner) to the computer 174.

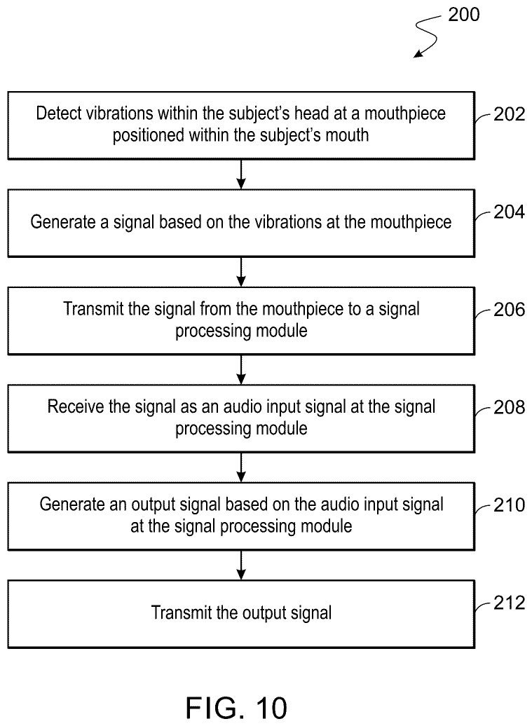

FIG. 10 is a flow chart illustrating an example method 200 of detecting an abnormal condition experienced by a subject (e.g., the patient 101 or another subject). In some embodiments, the method 200 includes a step 202 for detecting vibrations within the subject's head at a mouthpiece (e.g., the mouthpiece 102, 402) positioned within the subject's mouth. In some embodiments, the method 200 includes a step 204 for generating a signal based on the vibrations at the mouthpiece. In some embodiments, the method 200 includes a step 206 for transmitting the signal from the mouthpiece to a signal processing module (e.g., the signal processing module 104, 404; the signal processing console 130, 430; or the signal processing console 170). In some embodiments, the method 200 includes a step 208 for receiving the signal as an audio input signal at the signal processing module. In some embodiments, the method 200 includes a step 210 for generating an output signal based on the audio input signal at the signal processing module. In some embodiments, the method 200 includes a step 212 for transmitting the output signal.

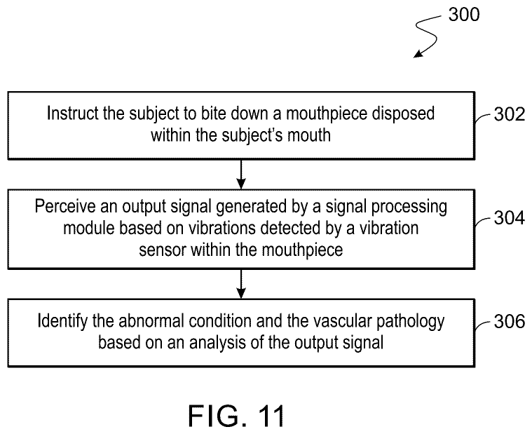

FIG. 11 is a flow chart illustrating an example method 300 of diagnosing a subject with an abnormal condition that is secondary to a vascular pathology. In some embodiments, the method 300 includes a step 302 for instructing the subject to bite down on a mouthpiece (e.g., the mouthpiece 102, 402) disposed within the subject's mouth. In some embodiments, the method 300 includes a step 304 for perceiving an output signal generated by a signal processing module (e.g., the signal processing module 104, 404; the signal processing console 130, 430; or the signal processing console 170) based on vibrations detected by a vibration sensor (e.g., the vibration sensor 110, 410) within the mouthpiece. In some embodiments, the method 300 includes a step 306 for identifying the abnormal condition and the vascular pathology based on an analysis (e.g., the frequency associated with the indicator 186 or another analysis) of the output signal.

The audio detection systems 100, 400 and other audio detection systems described below provide a robust tool for accurately detecting pulsatile tinnitus that results from (e.g., is secondary to) venous sinus stenosis. The disposable mouthpiece 102 of the audio detection systems 100, 400 may be embodied as an ergonomic bite block that facilitates comfortable holding of the mouthpiece 102 between a patient's teeth for a required time period during which vibrations are sensed at the mouthpiece 102. Furthermore, the mouthpiece 102 is designed to take advantage of bone conduction within the patient's head (e.g., via the patient's teeth), which allows a diagnostic investigation without subjecting the patient to an invasive procedure. The audio detection systems 100, 400 are also capable, advantageously, of outputting data (e.g., an analog audio output signal and a digital output signal) in manners that allow perception (e.g., listening and seeing, respectively) by both the patient and a clinician.

While the audio detection systems 100, 400 have been described and illustrated with respect to certain dimensions, sizes, shapes, arrangements, materials, and methods 200, 300, in some embodiments, an audio detection system that is otherwise similar in construction and/or function to the audio detection system 100 or the audio detection system 400 may include one or more different dimensions, sizes, shapes, arrangements, configurations, or materials, or be operated or otherwise utilized according to different methods. For example, while the audio detection systems 100, 400 have been described and illustrated as including wired connections among the signal processing module 104, 404 and the mouthpiece 102 and output device 106, in some embodiments, an audio detection system that is otherwise substantially similar in construction and function to the audio detection system 100 or the audio detection system 400 may alternatively include a wireless connection between a signal processing module and either or both of a mouthpiece and an output device. For example, in some embodiments, such a wireless connection may be implemented using a short-range wireless system, such as Bluetooth that operates using low-power radio waves on a frequency band between 2.400 GHz and 2.483.5 GHz.

While the audio detection systems 100, 400 have been described and illustrated as including a single signal processing module 104, 404 and a single output device 106, in some embodiments, an audio detection system that is otherwise substantially similar in construction and function to the audio detection system 100 or to the audio detection system 400 may include more than one signal processing module 104, 404 (e.g., such that the mouthpiece 102 is coupled to each signal processing module 104, 404) and/or may include more than one output device 106 (e.g., such that a signal processing module 104, 404 is coupled to each output device 106).

While the audio detection systems 100, 400 have been described and illustrated as including a mouthpiece 102 that includes a single vibration sensor 110, in some embodiments, an audio detection system that is otherwise substantially similar in construction and function to the audio detection system 100 or to the audio detection system 400 may include a mouthpiece that includes more than one vibration sensor 110. Such a mouthpiece may be otherwise substantially equivalent in construction and function to the mouthpiece 102. In embodiments that include multiple (e.g., two or more) vibration sensors 110, the respective vibration sensors 110 may be directional (e.g., having certain, respective physical orientations) and/or used to filter out signals within certain, respective frequency ranges.

Accordingly, other embodiments are within the scope of the following claims.

What is claimed is:

1. An audio detection system comprising:
a mouthpiece configured to:
detect vibrations occurring at a subject's head,
generate a signal based on the vibrations, and
transmit the signal; and
a signal processing module configured to:
receive the signal from the mouthpiece as an audio input signal,
generate an output signal based on the audio input signal, and
transmit the output signal.

2. The audio detection system of claim 1, wherein the mouthpiece comprises a bite block that is configured to be held between two or more of the subject's teeth and configured to conduct the vibrations from the two or more of the subject's teeth.

3. The audio detection system of claim 1, wherein the mouthpiece comprises a bone conduction device.

4. The audio detection system of claim 1, wherein the mouthpiece comprises a vibration sensor.

5. The audio detection system of claim 4, wherein the vibration sensor is configured to detect the vibrations as an analog signal.

6. The audio detection system of claim 4, wherein the vibration sensor is formed on an integrated circuit.

7. The audio detection system of claim 4, wherein the mouthpiece comprises an electrical cable that electrically connects the vibration sensor to the signal processing module.

8. The audio detection system of claim 5, wherein the output signal has a frequency within a range of about 100 Hz to about 10,000 Hz.

9. The audio detection system of claim 1, wherein the signal processing module comprises a benchtop console.

10. The audio detection system of claim 1, wherein the signal processing module comprises a handheld console.

11. The audio detection system of claim 1, wherein the output signal comprises a digital output signal.

12. The audio detection system of claim 11, wherein the signal processing module is configured to store the digital output signal as one or more recorded sounds.

13. The audio detection system of claim 11, wherein the signal processing module is configured to transmit the digital output signal to a display device for display in graphical form.

14. The audio detection system of claim 11, wherein the signal processing module is configured to convert the digital output signal to an analog audio output signal and transmit the analog audio output signal to an output device.

15. The audio detection system of claim 14, further comprising the output device.

16. The audio detection system of claim 15, wherein the output device comprises a headphone.

17. The audio detection system of claim 15, wherein the output device comprises a speaker.

18. The audio detection system of claim 1, wherein the signal processing module is configured to generate an indicator associated with the output signal and corresponding to an abnormal condition of the ear that is secondary to an underlying vascular pathology.

19. The audio detection system of claim 18, wherein the abnormal condition comprises pulsatile tinnitus.

20. The audio detection system of claim 19, wherein the underlying vascular pathology comprises cerebral venous sinus stenosis (CVSS).

21. A method of detecting an abnormal condition experienced by a subject, the method comprising:
detecting vibrations within the subject's head at a mouthpiece positioned within the subject's mouth;
generating a signal based on the vibrations at the mouthpiece;
transmitting the signal from the mouthpiece to a signal processing module;
receiving the signal as an audio input signal at the signal processing module;
generating an output signal based on the audio input signal at the signal processing module; and
transmitting the output signal.

22. The method of claim 21, wherein the mouthpiece comprises a bite block that is held between two or more of the subject's teeth and configured to conduct the vibrations from the two or more of the subject's teeth.

23. The method of claim 21, wherein the mouthpiece comprises a vibration sensor, and wherein the method further comprises detecting the vibrations as an analog signal at the vibration sensor.

24. The method of claim 21, wherein the output signal comprises a digital output signal.

25. The method of claim 24, further comprising:
storing the digital output signal as one or more recorded sounds; and/or
transmitting the digital output signal to a display screen and displaying a representation of the digital output signal on the display screen for observation by a viewer.

26. The method of claim 24, further comprising:
converting the digital output signal to an analog audio output signal; and
transmitting the analog audio output signal to an output device for perception by a listener.

27. The method of claim 26, wherein the output device comprises a headphone or a speaker.

28. The method of claim 21, further comprising outputting an indicator associated with the output signal to a display screen, wherein the indicator corresponds to the abnormal condition and a vascular pathology that underlies the abnormal condition.

29. The method of claim 28, wherein the abnormal condition comprises pulsatile tinnitus, and wherein the vascular pathology comprises cerebral venous sinus stenosis (CVSS).

30. The method of claim 29, wherein the indicator comprises one or more frequencies of the output signal.
